(19) United States
(12) Patent Application Publication
(10) Pub. No.: US 2024/0252445 A1
(43) Pub. Date: Aug. 1, 2024

(54) COMPOSITIONS AND METHODS OF PROMOTING ACCUMULATION OF THERAPEUTIC AND DIAGNOSTIC AGENTS IN THE HEART

(71) Applicant: CASE WESTERN RESERVE UNIVERSITY, Cleveland, OH (US)

(72) Inventors: Andrei Maiseyeu, Shaker Heights, OH (US); Lauren Elizabeth Switala, Cleveland, OH (US); Lin Di, Cleveland, OH (US)

(21) Appl. No.: 18/293,216

(22) PCT Filed: Jul. 29, 2022
(86) PCT No.: PCT/US2022/038816

§ 371 Date: Jan. 29, 2024

Related U.S. Application Data
(60) Provisional application No. 63/227,092, filed on Jul. 29, 2021.

(51) Int. Cl. A61K 9/51; A61K 9/107; A61K 35/28; A61K 48/00; B82Y 5/00; C12N 15/86

(52) U.S. Cl. CPC A61K 9/5153; A61K 9/1075; A61K 35/28; A61K 48/0041; C12N 15/86; B82Y 5/00; C12N 2750/14143

(57) ABSTRACT

A composition for use in promoting accumulation and/or uptake of a systemically delivered therapeutic agent and/or diagnostic agent in a heart of a subject in need thereof includes administering a plurality of poly(L-lactic co-glycolic acid) (PLLGA) nanoparticles effective to promote accumulation and/or uptake of the therapeutic agent and/or diagnostic agent in the heart.

### RELATED APPLICATION

This application claims priority from U.S. Provisional Application No. 63/227,092, filed Jul. 29, 2021, the subject matter of which is incorporated herein by reference in its entirety.

### BACKGROUND

Adeno-associated viral (AAV) gene therapy has seen accelerated clinical development in the last ten years, after some setbacks in the 1990s and early 2000s. Today, AAV gene therapy constructs have been designed and tested in almost every therapeutic area, for genetic and non-genetic conditions, including cardiac, such as heart failure and a variety of cardiomyopathies. AAV constructs have been employing several common AAV serotypes that exhibit cardiac muscle tropism, such as AAV6, AAV8, AAV9, AAVrh74, and AAVrh10. With respect to specific transgenes (cargo), gene transfer, gene editing, RNA interference, and vectorized therapeutic proteins have been tested by different academic labs and biopharma companies. The overwhelming majority of AAV constructs for cardiac applications are delivered intravenously, i.e., systemically. As is expected for systemic delivery of AAVs, the majority of viral vectors deposit to the liver, while a small percentage reaches the heart and other organs of interest. As a result of this preferential accumulation in the liver, systemic AAV gene therapies have several problems that the biopharma industry is very interested in solving. Targeted delivery to organs of interest is desirable because it could improve efficacy, safety, and cost of production of AAV gene therapy products.

### SUMMARY

Embodiments described herein relate to compositions and methods of promoting accumulation and/or uptake of a therapeutic agent and/or diagnostic agent in a heart of a subject in need thereof, and, particularly, relates to compositions and methods of treating and/or diagnosing heart pathologies.

We found that systemic administration (e.g., intravenous or parenteral injection) of a precision-engineered nano-adjuvant, which includes poly(L-lactic co glycolic acid) (PLLGA) (or ePL), in combination with a therapeutic agent and/or diagnostic agent to a subject can block uptake of the therapeutic agent and/or diagnostic agent in major non-cardiac organs of the subject, such as the brain, muscle, liver, lung, and adipose tissue, and promote accumulation, uptake, targeting and/or delivery of the therapeutic agent and/or diagnostic agent to the heart of the subject. Systemic administration of PLLGA nanoparticles was found to regulate glucose uptake in the heart and thus can increase the affinity of glycosylated therapeutic agents and diagnostic agents to upregulated cardiac GLUT transporters, highly expressed in cardiomyocytes, and other molecules, such as the insulin receptor. Systemic administration of PLLGA nanoparticles was also found to downregulate PI3K signaling in the liver and lower the hepatic uptake of glycosylsted therapeutic agents and diagnostic agents.

We further found that systemic administration of PLLGA nanoparticles to a subject can promote robust heart delivery of diverse entities/vehicles including a) small molecules, b) nanoparticles; c) viruses (AAV and Lenti); d) antisense oligonucleotides (ASO); e) proteins (including therapeutic mAbs); f) engineered and non-engineered cells, such as T-cells and stem cells, at exceptionally high heart-to-liver accumulation ratios of the entities/vehicles delivered in combination with the PLLGA nanoparticles and at orders of magnitude higher than delivery without the PLLGA nanoparticles. As such, systemic administration of PLLGA nanoparticles can provide a universal mode to promote delivery to the heart of potentially any therapeutic agent and/or diagnostic agent.

Accordingly, in some embodiments, a composition for use in promoting accumulation and/or uptake of a systemically delivered therapeutic agent and/or diagnostic agent in a heart of a subject in need thereof can include a plurality of poly(L-lactic co-glycolic acid) (PLLGA) nanoparticles effective to promote accumulation of the therapeutic agent and/or diagnostic agent to the heart.

In some embodiments, the therapeutic agent and/or diagnostic agent is not encapsulated by or conjugated to the PLLGA nanoparticles. In other words the PLLGA nanoparticles do not include or are free of the therapeutic agent that is systemically delivered to the subject and whose accumulation and/or uptake in the heart is promoted or enhanced by the administration of the PLLGA nanoparticles to the subject.

In some embodiments, the therapeutic agent and/or diagnostic agent can be delivered to the subject in the same formulation or composition as the composition including the PLLGA nanoparticles or separately from or in separate formulations than the composition including the PLLGA nanoparticles.

In some embodiments, the PLLGA nanoparticles can have an average diameter of about 50 nm to about 600 nm, about 75 nm to about 500 nm, or about 100 nm to about 400 nm, for example about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm or any number therebetween.

In some embodiments, the PLLGA of the PLLGA nanoparticles can have a molecular weight of about 15 kDa to about 200 kDa, about 60 kDa to about 180 kDa, or about 90 kDa to about 150 kDa, for example, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 160 kDa, about 170 kDa, about 180 kDa, about 190 kDa, about 200 kDa, or any number therebetween.

In other embodiments, the PLLGA can have an inherent viscosity as measured in CHCL₃ at 20° C. of about 0.30 dL/g to about 0.90 dL/g, about 0.35 dL/g to about 0.85 dL/g, about 0.40 dL/g to about 0.80 dL/g, about 0.45 dL/g to about 0.75 dL/g, or about 0.50 dL/g to about 0.70 dL/g, for example, about 0.30 dL/g, about 0.35 dL/g, about 0.40 dL/g, about 0.45 dL/g, about 0.50 dL/g, about 0.50 dL/g, about 0.55 dL/g, about 0.60 dL/g, about 0.65 dL/g, about 0.70 dL/g, about 0.75 dL/g, about 0.80 dL/g, about 0.85 dL/g, about 0.90 dL/g or any number therebetween.

In some embodiments, the PLLGA comprises about 60% L-lactic acid to about 85% L-lactic acid, about 60% L-Lactic acid to about 75% L-lactic acid, or about 60% L-lactic acid to about 70% L-lactic acid.

In other embodiments, the PLLGA comprises about 40% glycolic acid to about 15% glycolic acid, about 40% glycolic acid to about 25% glycolic acid, or about 40% glycolic acid to about 30% glycolic acid.

In some embodiments, the PLLGA nanoparticles comprise PLLGA micelles. The PLLGA nanoparticles or micelles can be formulated from PLLGA, a solvent or liquid carrier, and a surfactant. The surfactant can include, for example, poly(vinyl alcohol) (PVA), vitamin E d-α-tocopheryl polyethylene glycol 1000 succinate (vitamin E TPGS), or phosphatidylglycerol.

In some embodiments, the PLLGA nanoparticles can be PEGylated at an amount effective to modulate uptake and/or accumulation of the therapeutic agent and/or diagnostic agent in major non-cardiac organs of the subject, such as the brain, muscle, liver, lung, and adipose tissue, and accumulation, uptake, targeting and/or delivery of the therapeutic agent and/or diagnostic agent to the heart of the subject.

In other embodiments, the PLLGA nanoparticles are free of PEG.

The therapeutic agent and/or diagnostic agent can be naturally or synthetically glycosylated and/or include, be complexed with, and/or be conjugated to a carbohydrate, such as glucose or a glucose analog, to promote targeting and/or delivery of the therapeutic agent and/or diagnostic agent to heart by the PLLGA nanoparticles. In certain embodiment, the naturally or synthetically glycosylated therapeutic agent and/or diagnostic agent can target upregulated cardiac GLUT transporters and other molecules, such as the insulin receptor.

In some embodiments, the therapeutic agent can include any agent that can be used to treat and/or prevent a cardiac disease or disorder and/or disease or disorder that affects cardiac tissue. Such therapeutic agents can potentially be naturally or synthetically glycosylated and/or include, be complexed with, and/or be conjugated to a carbohydrate, such as glucose or a glucose analog, to promote targeting and/or delivery of the therapeutic agent to heart. Examples of therapeutic agents include small molecules, nanoparticles, therapeutic viruses or viral vectors, oligonucleotides, antisense oligonucleotides, gene editors, proteins, antibodies and fragments thereof, engineered and non-engineered cells.

In some embodiments, the therapeutic agent can include a viral vector. The viral vector can be used to deliver or encode at least one interfering RNA (e.g., siRNA), gene editing proteins and guide RNA (e.g., CRISPR/CAS9), and therapeutic proteins and genes. In certain embodiments the viral vector can exhibit cardiac muscle tropism.

In some embodiments, the viral vector is an adeno-associated viral vector (AAV). AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the methods and compositions described herein can be selected by a skilled person in the art based on the efficiency of delivery, tissue tropism, and immunogenicity. AAV serotypes identified from rhesus monkeys, e.g., rh.8, rh.10, rh.39, rh.43, and rh.74, are also contemplated in the compositions and methods described herein. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

In some embodiments, the adeno-associated viral vectors can include at least one of AAV1, AAV2, AAV6, AAV8, AAV9, AAVrh74, AAVrh10, AAV5, AAV7, AAVS3, AAVHSC, AAV2.7m8, AAV-LK03, AAV8/Olig001, AAV218, AAVhu37, AAV2tYF, AAVh1, AAVhu68, AAVrh.8, AAVrh9, AAV.PHP.B., AAV.PHP.eB, AAV.PHP.S, AAV/BBB, AAV-DJ, AAVr3.45, AAV-sh10, AAV2(Y444F), AAV4, AAV-RPF2, or AAV3b. It will be appreciated that other viral vectors can also be used in the methods described herein including lentivirus vectors, such as vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV), and adenoviral vectors.

In some embodiments, the viral vector can include cDNA that encodes at least one interfering RNA, gene editing proteins and guide RNA (e.g., CRISPR/CAS9), or therapeutic proteins. For example, the viral vector can include cDNA that encodes at least one cardiac protein selected from Titin, Lamin-A/C, Myosin 7 Heavy Chain, Myosin 6 Heavy Chain, Sodium Channel Protein Type 5 alpha subunit, Cardiac-Type Myosin Binding Protein C, Cardiac Muscle Troponin T, RNA-Binding Protein 20, Cardiac Troponin I, Regulatory Light Chain of Cardiac Myosin beta, Myosin Light Chain Ventricular Isoform, Plakophilin 2, Desmoplakin, Desmoglein, LAMP2B, Desmocolin 2, Junction Placoglobin, adenylyl cyclase (AC) 6 (AC6), sarco/endoplasmic reticulum (SR) Ca²⁺-ATPase (SERCA2a), SUMO1, S100A1, Ilc, VEGF-A, VEGF-B, β-adrenergic receptor kinase-ct, urocortins, B-cell lymphoma 2 (Bcl2)-associated anthanogene-3 (BAG3), Heme Oxygenase-1, anti-fribrotic agents, anti-inflammatory agents, or anti-hypertrophic agents.

It will be appreciated that the viral vector can encode other therapeutic proteins or genes as well as proteins and genes that provide an agnostic effect including an anti-hypertrophic, anti-inflammatory, and anti-fibrotic effect.

In other embodiments, the therapeutic agent can include cells, such as pluripotent cells, stem cells, or T-cells. The cells can be genetically engineered or non-engineered. In one example, the cells can include a plurality of stem cells, such as mesenchymal stem cells (MSCs).

In some embodiments, the diagnostic agent can include any agent or composition detectable by spectroscopic, photochemical, biochemical, immunochemical, electrical, optical, chemical or similar methods. Such diagnostic agents can include nucleotides (labeled or unlabeled), polymers, sugars, peptides, proteins, antibodies, chemical compounds, conducting polymers, binding moieties, mass tags, colorimetric agents, light emitting agents, chemiluminescent agents, light scattering agents, fluorescent agents, radioactive agents, charge agents (electrical or magnetic charge), volatile agents, biomolecules (e.g., members of a binding pair antibody/antigen, antibody/antibody, antibody/antibody fragment, antibody/antibody receptor), chemical reactive agents, infrared and near infrared agents, microparticles or nanoparticles, enzymes, and chelating agents. Such agents can be naturally or synthetically glycosylated and/or include, be complexed with, and/or be conjugated to a carbohydrate, preferably glucose or a glucose analog, to promote targeting and/or delivery of the diagnostic agent to the heart, by, for example, upregulated cardiac GLUT transporters and other molecules, such as the insulin receptor

In some embodiments, the diagnostic agent is a radiopharmaceutical. The radiopharmaceutical can include at least one of an inorganic tracer, radio metal ions, small organic tracers, or radiometal complex tracers. The radiopharmaceutical can be naturally or synthetically glycosylated and/or include, is complexed with, and/or conjugated to a carbohydrate, such as glucose or glucose analog.

Other embodiments described herein relate to a method of promoting accumulation and/or uptake of a therapeutic agent and/or diagnostic agent in a heart of a subject in need thereof. The method can include systemically administering to the subject a therapeutic agent and/or diagnostic agent and an amount of the composition including the PLLGA nanoparticles effective to promote accumulation and/or uptake of the therapeutic agent and/or diagnostic agent in the heart.

In some embodiments, the composition including the PLLGA nanoparticles can be administered to the subject at an amount effective to inhibit systemic uptake of the therapeutic agent and/or diagnostic agent in organs other than the heart. The organs other than the heart can include at least one of the brain, non-cardiac muscle, liver, lung, adipose tissue, and other non-heart organs.

In some embodiments, the therapeutic agent and/or diagnostic agent can be administered to the subject within 6 hours, within 5 hours, within 4 hours, within 3 hours, within 2 hours, or simultaneously with administration of the PLLGA.

In some embodiments, the composition, the therapeutic agent, and/or the diagnostic agent is administered to the subject by at least one of cutaneous, subcutaneous, intraperitoneal, intramuscular, intrasternal injection, intravenous, intracoronary, intramyocardial, or intra-arterial administration.

In some embodiments, the subject has or is at risk of a cardiac disease or disorder. For example, the subject can have or be at risk of myocardial infarction, myocardial ischemia/reperfusion injury, myocardial surgery, myocardial transplantation, chronic ischemia, arrhythmia, congestive heart failure, cardiomyopathy either as a stand-alone condition or condition associated with various disorders, such as hypertension, ischemic heart disease, cardiotoxicity, myocarditis, thyroid disease, viral infection, gingivitis, drug abuse, alcohol abuse, periocarditis, atherosclerosis, vascular disease, hypertrophic cardiomyopathy, acute myocardial infarction or previous myocardial infarction, left ventricular systolic dysfunction, coronary bypass surgery, starvation, an eating disorder, or a genetic defect.

In other embodiments, the cardiac disease or disorder can include at least one of Heart failure with reduced ejection fraction (HFrEF), Heart failure with preserved ejection fraction (HFpEF), Heart failure with mid-range ejection fraction (HFmrEF), hypertrophic cardiomyopathy (HCM), arrhythmogenic right ventricular cardiomyopathy (ARVC), dilated cardiomyopathy (DCM), diabetic cardiomyopathy (DbCM), Brugada Syndrome, Barth syndrome, arrhythmogenic left ventricular cardiomyopathy (LDAC or ALVC), Ventricular fibrillation, Ventricular tachycardia, Left Ventricular Non Compaction (LVNC), catecholaminergic polymorphic ventricular tachycardia (CPVC), Paroxysmal familial ventricular fibrillation (PFVF), Naxos disease, Carvajal syndrome, Wolff-Parkinson-White syndrome, Fabry disease, LEOPARD syndrome, Noonan syndrome, Anderson-Fabry disease, Familial amyloidosis, Kearns Sayre syndrome, MELAS syndrome, Becker MD, Duchenne MD, Emery-Dreifuss/Limb-Girdle MD, Friedreich's ataxia, Myotonic dystrophy, Down syndrome, AMPK mediated glycogenic storage, Pompe disease, Danon disease, Niemann-Pick, Refsum disease, or Chagas disease.

Other embodiments described herein relate to a method of treating a subject that has or is at risk of a cardiac disease or disorder in need thereof. The method can include systemically administering to the subject a cardiac therapeutic agent and an amount of the composition including the PLLGA nanoparticles effective to promote delivery of the cardiac therapeutic agent to the heart.

Still other embodiments relate to a method of imaging a heart of a subject in need thereof. The method can include systemically administering to the subject a diagnostic agent and an amount of the composition including the PLLGA effective to promote delivery of diagnostic agent to the heart.

In some embodiments, the method can further include detecting the diagnostic agent in the heart by at least one of positron emission tomography (PET) imaging or single-photon emission computed tomography (SPECT) imaging.

### BRIEF DESCRIPTION OF THE DRAWINGS

FIG. 1 illustrates a schematic summary of heart targeting and liver de-targeting with ePL (PLLGA nanoparticles). Circled numbers refer to further sections describing each mechanism in detail.

FIG. 2 illustrates a schematic showing possible ePL (PLLGA) formulations varying in physiochemical properties.

FIGS. 3(A-D) illustrate ePL facilitates heart targeting of glycosylated nanoparticle fluorospheres (NPs). A) Optimized injection schedule to test fluorosphere NP biodistribution with ePL. B) Biodistribution imaging in various organs of fluorescently-labeled NPs 2 h after i.v. administration. C) Analysis of the fluorescence intensity allowed to determine organ-to-liver ratios as normalized to the organ fluorescence from non-injected animals. D) Fluorescence microscopy of heart sections from mice injected as indicated. The sections were stained with anti-cardiac troponin (CT3) antibodies (green) co-registered with endogenous NP fluorescence (red, arrow-heads). Nuclei were visualized after staining with DAPI (blue). BAT=brown adipose tissue. n=S-6 mice/gr.

FIGS. 4(A-D) illustrate ePL improves heart targeting of adeno-associated viruses (AAV). A) Immunofluorescence analysis of hearts and livers in ePL- or vehicle-injected mice five weeks after injection of a single dose of 0.5×10¹² vg/kg AAVrh74.CMV.eGFP. B) Western blot analysis of GFP protein in heart and liver lysates. Right: densitometry quantification. C) qPCR analysis of vector copy numbers (VCN) in various organs from AAVrh74-injected animals with and without ePL. D) Experiments as in C, but with animals injected with 0.5×10¹² vg/kg AAV1.CMV-eGFP. The VCN were obtained after normalization against a standard curve. n=3-6 mice/gr.

FIGS. 5(A-F) illustrate ePL-mediated delivery of mesenchymal stem cells (MSCs). A, B) Metabolic labeling efficiency of MSCs was tested through the reaction of azide-labeled MSCs and fluorescent DBCO-AlexaFluor 488 (DB-O-AF488). Only Ac4ManNAz-incubated MSCs were reactive with DB-O-AF488. C, D) MSCs were labeled with PKH26 and glycosylated with DBCO-glucose. E) MSC^{PKH,Gluc} were injected at 5e5 cells/mouse±ePL and excised organs were imaged 2 h p.i. F) Quantification of MSCs delivery using qPCR for Alu sequences and a standard curve. n=3 mice/gr.

FIGS. 6(A-E) illustrate ePL changes systemic glucose metabolism. A) A schematic depiction of blood glucose monitoring experiments in conscious unrestrained mice continuously infused with ePL. B) ePL infusion rate and blood glucose in the same experiments. C) A single dose of ePL 1 h prior imaging in WT mice changes ¹⁸F-fluorodeoxyglucose (FOG) uptake. High uptake signal is seen in the heart. Positron emission tomography (PET) images and quantification (D) are shown. n=3 mice/gr. E) Fluorescently-labeled glucose (NBDG) uptake in the heart and liver using the same injection scheme as in FIG. 3A (with perfusion). Simultaneous injection with unlabeled glucose demonstrated specificity of glucose-driven heart uptake.

FIGS. 7(A-D) illustrate ePL enhances PK. A) A scheme of LSECs-mediated uptake. B) Depletion of LSECs diminishes heart targeting by NP^{Gluc}; n=3 animals. C) AAV9+ePL had delayed blood clearance. D) AAV9+ePL had higher overall levels of luciferase transgene expression.

FIGS. 8(A-B) illustrate a single injection of ePL increases the binding of MALII and RCA lectins in the heart. n=3 mice per group, ANOVA with Tukey post hoc.

### DETAILED DESCRIPTION

For convenience, certain terms employed in the specification, examples, and appended claims are collected here. Unless defined otherwise, all technical and scientific terms used herein have the same meaning as commonly understood by one of ordinary skill in the art to which this application belongs.

The articles “a” and “an” are used herein to refer to one or to more than one (i.e., to at least one) of the grammatical object of the article. By way of example, “an element” means one element or more than one element.

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “pharmaceutical composition” refers to a formulation containing the disclosed compounds in a form suitable for administration to a subject. In a preferred embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, inhalational, and the like. Dosage forms for the topical or transdermal administration of a compound described herein includes powders, sprays, ointments, pastes, creams, lotions, gels, solutions, patches, nebulized compounds, and inhalants. In a preferred embodiment, the active compound is mixed under sterile conditions with a pharmaceutically acceptable carrier, and with any preservatives, buffers, or propellants that are required.

The term “Nanoparticle” as used herein is meant to include particles, spheres, capsules, and other structures having a length or diameter of about 1 nm to less than about 1 μm. For the purposes of this application, the terms “nanosphere”, “nanoparticle”, “nanoparticle construct”, “nanovehicle”, or “nanocapsule” are used interchangeably.

“Optional” or “optionally” means that the subsequently described circumstance may or may not occur, so that the description includes instances where the circumstance occurs and instances where it does not.

The term “or” as used herein should be understood to mean “and/or”, unless the context clearly indicates otherwise.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

All percentages and ratios used herein, unless otherwise indicated, are by weight.

The term “congestive heart failure” refers to impaired cardiac function that renders the heart unable to maintain the normal blood output at rest or with exercise, or to maintain a normal cardiac output in the setting of normal cardiac filling pressure. A left ventricular ejection fraction of about 40% or less is indicative of congestive heart failure (by way of comparison, an ejection fraction of about 60% percent is normal). Patients in congestive heart failure display well-known clinical symptoms and signs, such as tachypnea, pleural effusions, fatigue at rest or with exercise, contractile dysfunction, and edema. Congestive heart failure is readily diagnosed by well-known methods (see, e.g., “Consensus recommendations for the management of chronic heart failure.” Am. J. Cardiol., 83(2A):1A-38-A, 1999).

Relative severity and disease progression are assessed using well known methods, such as physical examination, echocardiography, radionuclide imaging, invasive hemodynamic monitoring, magnetic resonance angiography, and exercise treadmill testing coupled with oxygen uptake studies.

The term “ischemic heart disease” refers to any disorder resulting from an imbalance between the myocardial need for oxygen and the adequacy of the oxygen supply. Most cases of ischemic heart disease result from narrowing of the coronary arteries, as occurs in atherosclerosis or other vascular disorders.

The term “myocardial infarction” refers to a process by which ischemic disease results in a region of the myocardium being replaced by scar tissue.

The term “cardiac hypertrophy” is used in its ordinary meaning as understood by the medical community and is often associated with increased risk of morbidity and mortality. It generally refers to the process in which adult cardiac myocytes respond to stress through hypertrophic growth. Such growth is characterized by cell size increases without cell division or proliferation, assembling of additional sarcomeres within the cell to maximize force generation, and an activation of a fetal cardiac gene program.

The term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. The terms “heart failure,” “manifestations of heart failure,” “symptoms of heart failure,” and the like are used broadly to encompass all of the sequelae associated with heart failure, such as shortness of breath, pitting edema, an enlarged tender liver, engorged neck veins, pulmonary rales and the like including laboratory findings associated with heart failure.

The term “nonischemic cardiomyopathy” refers to a disease of the myocardium associated with mechanical or electrical dysfunction exhibiting inappropriate ventricular hypertrophy or dilation. Nonischemic cardiomyopathy may be either primary (confined to the heart) or secondary to systemic conditions.

Embodiments described herein relate to compositions and methods of promoting accumulation and/or uptake of a therapeutic agent and/or diagnostic agent in a heart of a subject in need thereof, and, particularly, relates to compositions and methods of treating and/or diagnosing heart pathologies.

We found that systemic administration (e.g., intravenous or parenteral injection) of a precision-engineered nano-adjuvant, which includes poly(L-lactic co glycolic acid) (PLLGA) nanoparticles (or ePL), in combination with a therapeutic agent and/or diagnostic agent to a subject can block uptake of the therapeutic agent and/or diagnostic agent in major non-cardiac organs of the subject, such as the brain, muscle, liver, lung, and adipose tissue, and promote accumulation, uptake, targeting and/or delivery of the therapeutic agent and/or diagnostic agent to the heart of the subject.

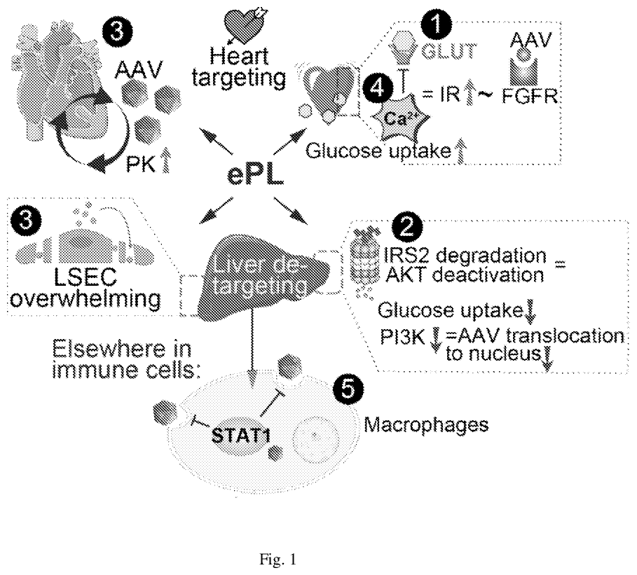

As illustrated schematically in FIG. 1, (1) systemic administration of PLLGA nanoparticles was found to regulate glucose uptake in the heart and thus can increase the affinity of glycosylated therapeutic agents and diagnostic agents to upregulated cardiac GLUT transporters, highly expressed in cardiomyocytes, and other molecules, such as insulin receptors. (2) Systemic administration of PLLGA nanoparticles was also found to downregulate PI3K signaling in the liver and lower the hepatic uptake of glycosylated therapeutic agents and diagnostic agents. (3) Because PLLGA nanoparticles can be micellar in solution, they can accumulate in and directly block the liver by temporarily overwhelming liver sinusoidal endothelial cells (LSECs), thus creating a therapeutic opportunity window for therapeutic agents and/or diagnostic agents to the heart. (4) PLLGA nanoparticles administered to isolated cardiomyocytes can reduce calcium transients in the cardiomyocytes through paracrine signaling, potentially promoting glucose uptake in the heart. (5) Systemic administration of PLLGA nanoparticles can upregulate a number of genes involved in viral replication, translational initiation, and other viral defense mechanisms. For example PLLGA nanoparticles can activate STAT1 through phosphorylation on Tyr701.

We further found that systemic administration of PLLGA nanoparticles can promote robust heart delivery of diverse entities/vehicles including a) small molecules, b) nanoparticles; c) viruses (AAV and Lenti); d) antisense oligonucleotides (ASO); e) proteins (including therapeutic mAbs); f) engineered and non-engineered cells, such as T-cells and stem cells, at exceptionally high heart-to-liver accumulation ratios of the entities/vehicles delivered in combination with the PLLGA nanoparticles and at orders of magnitude higher than delivery without the PLLGA nanoparticles. Accordingly, systemic administration of PLLGA can provide a universal mode to promote delivery to the heart of potentially any therapeutic agent and/or diagnostic agent.

A composition for use in promoting accumulation and/or uptake of a systemically delivered therapeutic agent and/or diagnostic agent in a heart of a subject in need thereof can include a plurality of poly(L-lactic co-glycolic acid) (PLLGA) nanoparticles effective to promote accumulation and/or uptake of the therapeutic agent and/or diagnostic agent in the heart.

The PLLGA nanoparticles of the composition may be uniform (e.g., being about the same size) or of variable size. Particles may be any shape (e.g., spherical or rod shaped), but are preferably made of regularly shaped material (e.g., spherical). Other geometries can include substantially spherical, circular, triangle, quasi-triangle, square, rectangular, hexagonal, oval, elliptical, rectangular with semi-circles or triangles and the like. Selection of suitable geometries are known in the art.

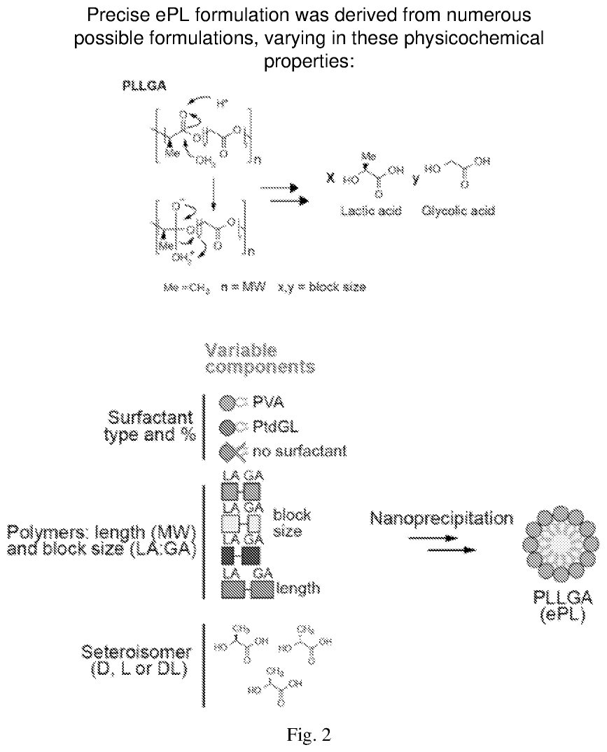

FIG. 2 illustrates a schematic showing possible PLLGA formulations varying in physiochemical properties. It was found that the interplay between the size of the nanoparticles, the ratio of L-lactic acid to glycolic acid, the molecular weight of the PLLGA, and inherent viscosity of the PLLGA can be varied and/or optimized to promote accumulation and/or uptake of systemically delivered therapeutic agents and/or diagnostic agents in the heart.

In some embodiments, the PLLGA nanoparticles can have a diameter effective upon systemic administration to promote accumulation and/or uptake of the therapeutic agent and/or diagnostic agent in the heart. For example, the average diameter of the PLLGA nanoparticles effective to promote accumulation and/or uptake of the therapeutic agent and/or diagnostic agent in the heart can be about 50 nm to about 600 nm, about 75 nm to about 500 nm, or about 100 nm to about 400 nm, for example about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm or any number therebetween.

In some embodiments, the PLLGA nanoparticles can have a distribution of diameters such that no more than about 10%, about 20%, about 30%, about 40%, or about 50% of the particles have a diameter greater than the average diameter noted above, and in some embodiments, such that no more than about 0.1%, 0.2%, 0.4%, 0.6%, 0.8%, 1%, 2%, 3%, 4%, 5%, 6%, 7%, 8%, 9%, 10%, 15%, 20%, or 25% of the PLLGA nanoparticles have a diameter greater than the average diameters noted above.

In some embodiments, the PLLGA of the PLLGA nanoparticles can have a molecular weight effective upon systemic administration to promote accumulation and/or uptake of the therapeutic agent and/or diagnostic agent in the heart. For example, the molecular weight of the PLLGA can be about 15 kDa to about 200 kDa, about 60 kDa to about 180 kDa, or about 90 kDa to about 150 kDa, for example, about 20 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 160 kDa, about 170 kDa, about 180 kDa, about 190 kDa, about 200 kDa, or any number therebetween.

In other embodiment, the PLLGA can have an inherent viscosity effective upon systemic administration to promote accumulation and/or uptake of the therapeutic agent and/or diagnostic agent in the heart. For example, the inherent viscosity as measured in CHCL₃ at 20° C. of the PLLGA can be about 0.30 dL/g to about 0.90 dL/g, about 0.35 dL/g to about 0.85 dL/g, about 0.40 dL/g to about 0.80 dL/g, about 0.45 dL/g to about 0.75 dL/g, or about 0.50 dL/g to about 0.70 dL/g, for example, about 0.30 dL/g, about 0.35 dL/g, about 0.40 dL/g, about 0.45 dL/g, about 0.50 dL/g, about 0.50 dL/g, about 0.55 dL/g, about 0.60 dL/g, about 0.65 dL/g, about 0.70 dL/g, about 0.75 dL/g, about 0.80 dL/g, about 0.85 dL/g, about 0.90 dL/g or any number therebetween.

In some embodiments, ratios of units of the L-lactic acid and glycolic acid can vary, forming a PLLGA having different properties, e.g., different degradation rates, different flexibility or mechanical properties. For example, an increase in the glycolic acid monomers relative to the L-lactic monomers can provide an accelerated or enhanced degradation of the PLLGA; whereas an increase in the L-lactic monomers relative to the glycolic acid monomers can provide an increase in mechanical strength to the PLLGA.

In some embodiments, the PLLGA can include about 60% L-lactic acid to about 85% L-lactic acid, about 60% L-lactic acid to about 75% L-lactic acid, or about 60% L-lactic acid to about 70% L-lactic acid. In other embodiments, the PLLGA can include about 40% glycolic acid to about 15% glycolic acid, about 40% glycolic acid to about 25% glycolic acid, or about 40% glycolic acid to about 30% glycolic acid.

In some embodiments, the ratio of the various glycolic acid or L-lactic monomers can vary along the chain of the PLLGA. One point of the chain of the PLLGA polymer can be heavy with one monomer while another point of the chain can be light with the same monomer. If a monofunctional initiator is used, and if the selected monomers have highly different reactivity ratios, then a gradient of composition can be generated as the monomers are consumed during the polymerization. In another methodology, such a PLLGA polymer can be prepared by so-called gradient polymerization wherein during the polymerization a first or second monomer is progressively added to the reactor containing all, or a portion of, the first monomer. Yet a third method is by introducing blocks of various ratios of the monomers into the chain of the PLLGA polymer.

Randomness of the PLLGA described herein can be measured by randomness index. Generally, a perfectly alternating co-polymer would have a degree of randomness of 1. Conversely, in some embodiments, the PLLGA can include all the repeating units of the monomers in two blocks, the L-lactic acid block and the glycolic acid block. Such a PLLGA would have a degree of randomness of 0. These are known as block copolymers.

In some embodiments, the PLLGA can have a degree of randomness ranging from above 0 to below 1, for example, about 0.01, about 0.02, about 0.05, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, or about 0.99. Generally, for a crystalline domain to develop, one usually needs a pentad (i.e., the same 5 repeat units or monomers in sequence). Therefore, in some embodiments, one factor to control the randomness of the PLLGA is to keep the repeat units or monomers in sequence in the terpolymer below 5, e.g., 1, 2, 3, or 4.

Randomness in a polymer can be readily determined by established techniques in the art. One such technique is NMR analysis ((see, e.g., J. Kasperczyk, Polymer, 37(2):201-203 (1996); Mangkorn Srisa-ard, et al., Polym Int., 50:891-896 (2001)).

Randomness of an amorphous PLLGA can be readily controlled or varied using techniques known in the art. For example, randomness in a batch reactor is controlled by polymerization temperature and type of solvent where the monomer reactivity ratios will change. For continuous reactors, it will also depend on monomer feed ratios and temperature. Secondarily, there is also a pressure effect on reactivity ratios. Monomers relative reactivity is also important, so you can control it by selecting monomers with similar or different reactivity.

In some embodiments, the PLLGA nanoparticles can include PLLGA micelles. The PLLGA nanoparticles or micelles can be formulated from PLLGA, a solvent or liquid carrier, and a surfactant. The surfactant can include, for example, poly(vinyl alcohol) (PVA), vitamin E d-α-tocopheryl polyethylene glycol 1000 succinate (vitamin E TPGS), or phosphatidylglycerol.

In some embodiments, the PLLGA nanoparticles can be prepared by dissolving the PLLGA and surfactant in an organic solvent. The organic phase can be poured into an aqueous solution and then emulsified using a high speed shear homogenizer. The organic solvent can be removed by either slow evaporation at ambient temperature and normal pressure under stirring, or by quick evaporation at reduced pressure. During the process, the nanodroplets solidify in the aqueous system. The resulting nanosuspension of PLLGA nanoparticles can be filtered through a filter. For storage a cryoprotecting agent can be added. The suspension can then be filled in vials, frozen, and subsequently freeze-dried.

In other embodiments, the PLLGA can be dissolved in an organic solvent. The aqueous solution can be added to the organic phase. The mixture can emulsified. The obtained emulsion can be added to an aqueous solution of a surfactant and then further emulsified. The resulting coarse emulsion can be passed through a high-pressure homogenizer. The homogenization step can be repeated several times to produce a stable emulsion. Then, the organic solvent can be removed by slow evaporation at ambient temperature and normal pressure. The obtained nanosuspension of PLLGA nanoparticles can be filtered through a filter. For storage, a cryoprotecting agent can be added, and the nanosuspension can be frozen, and then freeze-dried.

In some embodiments, the PLLGA nanoparticles can be modified with a polymer that can modulate the ability of the PLLGA nanoparticles to promote uptake and/or accumulation of the therapeutic agent and/or diagnostic agent in the heart. The polymer can include, for example, a polyethylene glycol (PEG). The PLLGA nanoparticles can be PEGylated at an amount effective to modulate uptake and/or accumulation of the therapeutic agent and/or diagnostic agent in major non-cardiac organs of the subject, such as the brain, muscle, liver, lung, and adipose tissue, and accumulation, uptake, targeting and/or delivery of the therapeutic agent and/or diagnostic agent to the heart of the subject. For example, PEGylation of the PLLGA nanoparticles can potentially reduce and/or delay degradation of the PLLGA nanoparticles upon systemic administration and/or reduce or sustain uptake and/or accumulation of the therapeutic agent and/or therapeutic agent to the heart.

In other embodiments, the PLLGA nanoparticles are free of a polymer, such as PEG, that can modulate the ability of the PLLGA nanoparticles to promote uptake and/or accumulation of the therapeutic agent and/or diagnostic agent in the heart.

In some embodiments, the PLLGA nanoparticles described herein can be provided in a pharmaceutical composition. Such a pharmaceutical composition may consist of the PLLGA nanoparticles alone, in a form suitable for administration to a subject, or the pharmaceutical composition may comprise PLLGA nanoparticles and one or more pharmaceutically acceptable carriers, one or more additional ingredients, one or more therapeutic agents and/or diagnostic agents described herein, or some combination of these.

The formulations of the pharmaceutical compositions described herein may be prepared by any method known or hereafter developed in the art of pharmacology. In general, such preparatory methods include the step of bringing the PLLGA nanoparticles into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, which are suitable for ethical administration to humans, it will be understood by the skilled artisan that such compositions are generally for administration to animals of all sorts. Modification of pharmaceutical compositions for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, animals including commercially relevant animals such as cattle, pigs, horses, sheep, cats, and dogs, birds including commercially relevant birds such as chickens, ducks, geese, and turkeys.

The compositions described herein may be administered via numerous routes, including, but not limited to, systemic routes or parenteral routes, such as direct injection intraarterial administration, intracoronary administration, intramyocardial administration, and/or intravenous administration. The route(s) of administration will be readily apparent to the skilled artisan and will depend upon any number of factors including the type and severity of the disorder being treated, the type and age of the veterinary or human patient being treated, and the like.

Parenteral administration of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition on or through a surgical incision, by application of the composition on or through a tissue-penetrating non-surgical wound, and the like. In particular, parenteral administration is contemplated to include, but is not limited to, cutaneous, subcutaneous, intraperitoneal, intramuscular, intrasternal injection, intravenous, intracoronary, intramyocardial, and intra-arterial.

Formulations of a pharmaceutical composition suitable for parenteral administration can include the PLLGA nanoparticles combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles. Such formulations may further comprise one or more additional ingredients including, but not limited to, suspending, stabilizing, or dispersing agents. In one embodiment of a formulation for parenteral administration, the therapeutic agent is provided in dry (i.e., powder or granular) form for reconstitution with a suitable vehicle (e.g., sterile pyrogen free water) prior to parenteral administration of the reconstituted composition.

The pharmaceutical compositions described herein may also be formulated so as to provide slow, prolonged or controlled release. In general, a controlled-release preparation is a pharmaceutical composition capable of releasing nanoparticle or microparticle constructs at a desired or required rate to maintain constant activity for a desired or required period of time.

In some embodiments, a pharmaceutical composition that includes the PLLGA nanoparticles can be systemically delivered, by for example, parenteral administration, in a unit dose. As used herein, a “unit dose” is discrete amount of the pharmaceutical composition having a predetermined amount of the activity. The amount of the activity is generally equal to the dosage, which would be administered to a subject or a convenient fraction of such a dosage such as, for example, one-half or one-third of such a dosage.

The pharmaceutical compositions may be prepared, packaged, or sold in the form of a sterile injectable aqueous or oily suspension or solution. This suspension or solution may be formulated according to the known art, and may comprise, in addition to the PLLGA nanoparticles, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

In some embodiments, the therapeutic agent and/or diagnostic agent can be combined, mixed, and/or provided with the composition that includes the PLLGA nanoparticles. In such a composition, it will be appreciated that the therapeutic agent and/or diagnostic agent is not encapsulated by or conjugated to the PLLGA nanoparticles. In other words the PLLGA nanoparticles do not include or are free of the therapeutic agent that is systemically delivered to the subject and whose accumulation and/or uptake in the heart is promoted or enhanced by the administration of the PLLGA nanoparticles to the subject.

In other embodiments, the therapeutic agent and/or diagnostic agent can be delivered to the subject separately from or in separate formulations than the composition including the PLLGA nanoparticles.

In some embodiments, the therapeutic agent and/or diagnostic agent can include at least one of small molecules, nanoparticles, therapeutic viruses or viral vectors, oligonucleotides, antisense oligonucleotides, gene editors, proteins, antibodies and fragments thereof, engineered and non-engineered cells. The therapeutic agent and/or diagnostic agent can be naturally or synthetically glycosylated and/or include, be complexed with, and/or be conjugated to a carbohydrate, such as glucose or a glucose analog, to promote uptake, accumulation, targeting and/or delivery of the therapeutic agent and/or diagnostic agent to heart. In certain embodiment, the glycosylated therapeutic agent and/or diagnostic agent can target upregulated cardiac GLUT transporters and other molecules, such as the insulin receptor.

In some embodiments, the therapeutic agent and/or diagnostic agent in combination with the PLLGA nanoparticles can be used to treat or diagnose a subject that has or is at risk of a cardiac disease or disorder and/or disease or disorder that affects cardiac tissue. For example, the subject can have or be at risk of myocardial infarction, myocardial ischemia/reperfusion injury, myocardial surgery, myocardial transplantation, chronic ischemia, arrhythmia, congestive heart failure, cardiomyopathy either as a stand-alone condition or condition associated with various disorders, such as hypertension, ischemic heart disease, cardiotoxicity, myocarditis, thyroid disease, viral infection, gingivitis, drug abuse, alcohol abuse, periocarditis, atherosclerosis, vascular disease, hypertrophic cardiomyopathy, acute myocardial infarction or previous myocardial infarction, left ventricular systolic dysfunction, coronary bypass surgery, starvation, an eating disorder, or a genetic defect.

In other embodiments, the cardiac disease or disorder can include at least one of Heart failure with reduced ejection fraction (HFrEF), Heart failure with preserved ejection fraction (HFpEF), Heart failure with mid-range ejection fraction (HFmrEF), hypertrophic cardiomyopathy (HCM), arrhythmogenic right ventricular cardiomyopathy (ARVC), dilated cardiomyopathy (DCM), diabetic cardiomyopathy (DbCM), Brugada Syndrome, Barth syndrome, arrhythmogenic left ventricular cardiomyopathy (LDAC or ALVC), Ventricular fibrillation, Ventricular tachycardia, Left Ventricular Non Compaction (LVNC), catecholaminergic polymorphic ventricular tachycardia (CPVC), Paroxysmal familial ventricular fibrillation (PFVF), Naxos disease, Carvajal syndrome, Wolff-Parkinson-White syndrome, Fabry disease, LEOPARD syndrome, Noonan syndrome, Anderson-Fabry disease, Familial amyloidosis, Kearns Sayre syndrome, MELAS syndrome, Becker MD, Duchenne MD, Emery-Dreifuss/Limb-Girdle MD, Friedreich's ataxia, Myotonic dystrophy, Down syndrome, AMPK mediated glycogenic storage, Pompe disease, Danon disease, Niemann-Pick, Refsum disease, or Chagas disease.

In some embodiments, the therapeutic agent used to treat and/or prevent a cardiac disease or disorder and/or disease or disorder that affects cardiac tissue can be administered to a subject that has or is at risk of a cardiac disease or disorder in combination with an amount of the composition including the PLLGA nanoparticles effective to promote delivery of the cardiac therapeutic agent to the heart. Such cardiac therapeutic agents can potentially be naturally or synthetically glycosylated and/or include, be complexed with, and/or be conjugated to a carbohydrate, such as glucose or a glucose analog, to promote uptake, accumulation, targeting and/or delivery of the therapeutic agent to the heart.

Examples of therapeutics agents that can potentially be naturally or synthetically glycosylated and/or include, be complexed with, and/or be conjugated to a carbohydrate, such as glucose or a glucose analog, to promote targeting and/or delivery of the therapeutic agent to heart include, but are not limited to, beta blockers, anti-hypertensives, cardiotonics, anti-thrombotics, vasodilators, hormone antagonists, inotropes, diuretics, endothelin antagonists, calcium channel blockers, phosphodiesterase inhibitors, ACE inhibitors, angiotensin type 2 antagonists and cytokine blockers/inhibitors, and HDAC inhibitors.

More specifically, the therapeutic agent can include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In other embodiments, the therapeutic agent that is used to treat and/or prevent a cardiac disease or disorder and/or disease or disorder that affects cardiac tissue can include a viral vector. The viral vector can potentially be naturally or synthetically glycosylated and/or include, be complexed with, and/or be conjugated to a carbohydrate, such as glucose or a glucose analog, to promote uptake, accumulation, targeting and/or delivery of the viral vector to the heart. The viral vector can be used to deliver or encode at least one interfering RNA (e.g., siRNA), gene editing proteins and guide RNA (e.g., CRISPR/CAS9), and therapeutic proteins and genes. In certain embodiments the viral vector can exhibit cardiac muscle tropism.

In some embodiments, the viral vector is an adeno-associated viral vector (AAV). AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the methods and compositions described herein can be selected by a skilled person in the art based on the efficiency of delivery, tissue tropism, and immunogenicity. AAV serotypes identified from rhesus monkeys, e.g., rh.8, rh. 10, rh.39, rh.43, and rh.74, are also contemplated in the compositions and methods described herein. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

In some embodiments, the adeno-associated viral vectors can include at least one of AAV1, AAV2, AAV6, AAV8, AAV9, AAVrh74, AAVrh10, AAV5, AAV7, AAVS3, AAVHSC, AAV2.7m8, AAV-LK03, AAV8/Olig001, AAV218, AAVhu37, AAV2tYF, AAVh1, AAVhu68, AAVrh.8, AAVrh9, AAV.PHP.B., AAV.PHP.eB, AAV.PHP.S, AAV/BBB, AAV-DJ, AAVr3.45, AAV-sh10, AAV2(Y444F), AAV4, AAV-RPF2, or AAV3b. It will be appreciated that other viral vectors can also be used in the methods described herein including lentivirus vectors, such as vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV), and adenoviral vectors.

In some embodiments, the viral vector can include cDNA that encodes at least one interfering RNA, gene editing proteins and guide RNA (e.g., CRISPR/CAS9), or therapeutic proteins. For example, the viral vector can include cDNA that encodes at least one cardiac protein selected from Titin, Lamin-A/C, Myosin 7 Heavy Chain, Myosin 6 Heavy Chain, Sodium Channel Protein Type 5 alpha subunit, Cardiac-Type Myosin Binding Protein C, Cardiac Muscle Troponin T, RNA-Binding Protein 20, Cardiac Troponin I, Regulatory Light Chain of Cardiac Myosin beta, Myosin Light Chain Ventricular Isoform, Plakophilin 2, Desmoplakin, Desmoglein, LAMP2B, Desmocolin 2, Junction Placoglobin, adenylyl cyclase (AC) 6 (AC6), sarco/endoplasmic reticulum (SR) Ca²⁺-ATPase (SERCA2a), SUMO1, S100A1, Ilc, VEGF-A, VEGF-B, β-adrenergic receptor kinase-ct, urocortins, B-cell lymphoma 2 (Bcl2)-associated anthanogene-3 (BAG3), Heme Oxygenase-1, anti-fribrotic agents, anti-inflammatory agents, or anti-hypertrophic agents.

It will be appreciated that the viral vector can encode other therapeutic proteins or genes as well as proteins and genes that provide an agnostic effect including an anti-hypertrophic, anti-inflammatory, and anti-fibrotic effect.

In still other embodiments, the therapeutic agent that is used to treat and/or prevent a cardiac disease or disorder and/or disease or disorder that affects cardiac tissue can include cells, such as pluripotent cells, stem cells, or T-cells. The cells can potentially be naturally or synthetically glycosylated and/or include, be complexed with, and/or be conjugated to a carbohydrate, such as glucose or a glucose analog, to promote uptake, accumulation, targeting and/or delivery of the cells to the heart. The cells can be genetically engineered or non-engineered. In one example, the cells can include a plurality of stem cells, such as mesenchymal stem cells (MSCs).

In some embodiments, the diagnostic agent can include an agent that is used to detect and/or monitor a cardiac disease or disorder and/or disease or disorder that affects cardiac tissue. The diagnostic agent can be administered to a subject that has or is at risk of a cardiac disease or disorder in combination with an amount of the composition including the PLLGA nanoparticles effective to promote delivery of the diagnostic agent to the heart. Such diagnostic agents can potentially be naturally or synthetically glycosylated and/or include, be complexed with, and/or be conjugated to a carbohydrate, such as glucose or a glucose analog, to promote uptake, accumulation, targeting and/or delivery of the diagnostic agent to the heart.

In some embodiments, the diagnostic agent can include any agent or composition detectable by spectroscopic, photochemical, biochemical, immunochemical, electrical, optical, chemical or similar methods. Such diagnostic agents can include nucleotides (labeled or unlabeled), polymers, sugars, peptides, proteins, antibodies, chemical compounds, conducting polymers, binding moieties, mass tags, colorimetric agents, light emitting agents, chemiluminescent agents, light scattering agents, fluorescent agents, radioactive agents, charge agents (electrical or magnetic charge), volatile agents, biomolecules (e.g., members of a binding pair antibody/antigen, antibody/antibody, antibody/antibody fragment, antibody/antibody receptor), chemical reactive agents, infrared and near infrared agents, microparticles or nanoparticles, enzymes, and chelating agents. Such agents can be glycosylated and/or include, be complexed with, and/or be conjugated to a carbohydrate, preferably glucose or a glucose analog, to promote targeting and/or delivery of the diagnostic agent to the heart, by, for example, upregulated cardiac GLUT transporters and other molecules, such as the insulin receptor

In some embodiments, the diagnostic agent is a radiopharmaceutical. The radiopharmaceutical can include at least one of an inorganic tracer, radio metal ions, small organic tracers, or radiometal complex tracers. The radiopharmaceutical can be glycosylated and/or include, is complexed with, and/or conjugated to a carbohydrate, such as glucose or glucose analog. In other embodiments, the diagnostic agent can be detectable in the heart by at least one of positron emission tomography (PET) imaging or single-photon emission computed tomography (SPECT) imaging.

In some embodiments, the therapeutic agent and/or diagnostic agent can be administered to the subject at or within a duration of time of administration of the PLLGA nanoparticles effective to promote uptake and/or accumulation of the therapeutic agent and/or diagnostic agent within the heart. The duration of time can be within 6 hours, within 5 hours, within 4 hours, within 3 hours, within 2 hours, or simultaneously with administration of the PLLGA. For example, the therapeutic agent and/or diagnostic agent and the PLLGA nanoparticles may be administered simultaneously with, less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part.

In certain embodiments, the therapeutic agent and/or diagnostic agent and the PLLGA nanoparticles can be cyclically administered. Cycling therapy involves the administration of a PLLGA nanoparticles for a period of time, followed by the administration of the therapeutic agent and/or diagnostic agent for a period of time and repeating this sequential administration, e.g., the cycle, in order to improve the efficacy of the therapeutic agent and/or diagnostic agent. In certain embodiments, the administration of the combination therapy may be repeated and the administrations may be separated by at least 1 day, 2 days, 3 days, 5 days, 10 days, 15 days, 30 days, 45 days, 2 months, 75 days, 3 months, or at least 6 months.

In some embodiments, the composition including the PLLGA nanoparticles can be administered to the subject at an amount effective to inhibit systemic uptake of the therapeutic agent and/or diagnostic agent in organs other than the heart. The organs other than the heart can include at least one of the brain, non-cardiac muscle, liver, lung, adipose tissue, and other non-heart organs.

Doses of the PLLGA nanoparticles for promoting accumulation and/or uptake of the therapeutic agent and/or diagnostic agent in the heart as well as dosage of the therapeutic agent and/or diagnostic agent for treating and/or diagnosing a particular patient can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The doses of the PLLGA nanoparticles and therapeutic agent and/or diagnostic agent administered to a patient can be sufficient to effect a beneficial therapeutic response in the patient over time, or, e.g., to reduce symptoms, or other appropriate activity, depending on the application. The doses can be determined by the efficacy of the particular formulation, and the activity, stability or serum half-life of the PLLGA nanoparticles and therapeutic agent and/or diagnostic agent employed and the condition of the patient, as well as the body weight or surface area of the patient to be treated. The size of the dose can also be determined by the existence, nature, and extent of any adverse side-effects that accompany the administration of a particular composition in a particular patient.

Optimal precision in achieving concentrations of the therapeutic regimen (e.g., a pharmaceutical composition comprising the PLLGA nanoparticles and therapeutic agent and/or diagnostic agent) within the range that yields maximum efficacy with minimal toxicity may require a regimen based on the kinetics of the pharmaceutical composition's availability to one or more target sites. Distribution, equilibrium, and elimination of a pharmaceutical composition may be considered when determining the optimal concentration for a treatment regimen. Generally, the pharmaceutical compositions of the present invention may be administered in a manner that maximizes efficacy and minimizes toxicity.

Moreover, the dose administration of PLLGA nanoparticles and therapeutic agent and/or diagnostic agent may be optimized using a pharmacokinetic/pharmacodynamic modeling system. For example, one or more dosage regimens may be chosen and a pharmacokinetic/pharmacodynamic model may be used to determine the pharmacokinetic/pharmacodynamic profile of one or more dosage regimens. Next, one of the dosage regimens for administration may be selected which achieves the desired pharmacokinetic/pharmacodynamic response based on the particular pharmacokinetic/pharmacodynamic profile.

Doses of a pharmaceutical composition that includes the PLLGA nanoparticles for promoting accumulation and/or uptake of the therapeutic agent and/or diagnostic agent in the heart can optionally include 0.0001 μg to 1,000 mg/kg/administration, or 0.001 μg to 100.0 mg/kg/administration, from 0.01 μg to 10 mg/kg/administration, from 0.1 μg to 10 mg/kg/administration, including, but not limited to, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53,54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration or any range, value or fraction thereof.

As a non-limiting example, treatment of humans or animals can be provided as a onetime or periodic dose of a composition of the present invention 0.1 ng to 100 mg/kg such as 0.0001, 0.001, 0.01, 0.1 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

The doses of PLLGA nanoparticles for promoting accumulation and/or uptake of the therapeutic agent and/or diagnostic agent in the heart as well as dosage of the therapeutic agent and/or diagnostic agent for treating and/or diagnosing a particular patient disclosed herein may be adjusted when combined to achieve desired effects. On the other hand, dosages of the PLLGA nanoparticles for promoting accumulation and/or uptake of the therapeutic agent and/or diagnostic agent in the heart as well as dosage of the therapeutic agent and/or diagnostic agent for treating and/or diagnosing a particular patient may be independently optimized and combined to achieve a synergistic result wherein the pathology is reduced more than it would be if either were used alone.

The following example is included to demonstrate different embodiments of the invention. It should be appreciated by those of skill in the art that the techniques disclosed in the example, which follow represent techniques discovered by the inventors to function well in the practice of the claimed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the claims.

### EXAMPLE

The following example describes the use of ePL (PLLGA nanoparticles) in promoting delivery of therapeutic modalities, such as viral vectors and cells, to the heart. As exemplified using AAVrh74, superior delivery to the heart using ePL directly impacts dose and manufacturing cost of AAV gene therapy. It reduces the dose by at least an order of magnitude, and as a result decreases cost of manufacturing and increases safety of the treatment. Safety and manufacturing costs directly impact the bottom line

Enhanced Delivery to the Heart with ePL

We discovered a synthetic biodegradable polymer that exhibited unique biological and physiological responses upon i.v. injection in mice. Guided by ePL actions in vivo, we largely deciphered its mechanism of action and, along the way, serendipitously found the ability of ePL to divert the uptake of AAVs to the heart. The enhanced heart transduction occurs simultaneously with significant blockade of AAV uptake in other organs, including liver, spleen, kidneys and skeletal muscle. If ePL is administered immediately prior to an AAV injection, a drastic amplification of the delivery to the heart occurs within the first 2 h of ePL administration. Such an efficient delivery mechanism allowed us to reduce the AAV dose to 5×10¹¹ vg/kg, which is 20-100 times lower than previously used AAV doses for heart targeting.

Enhanced Delivery with ePL

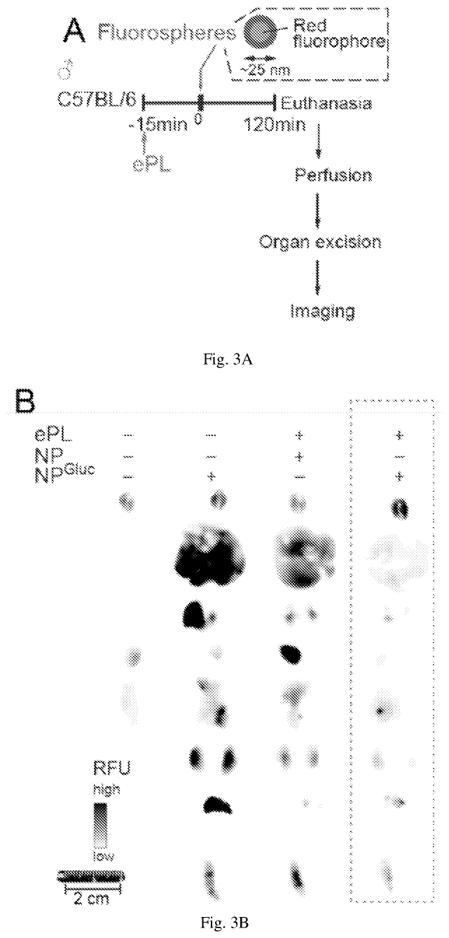

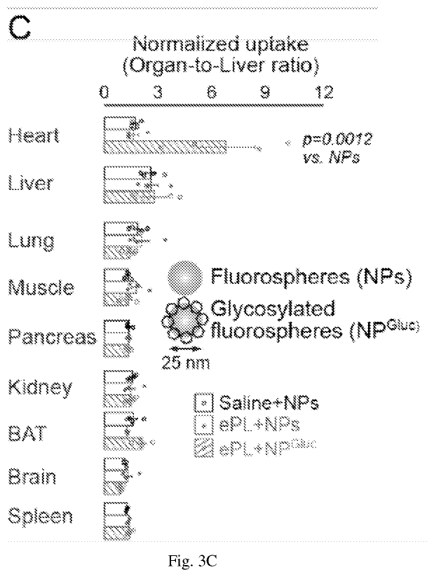

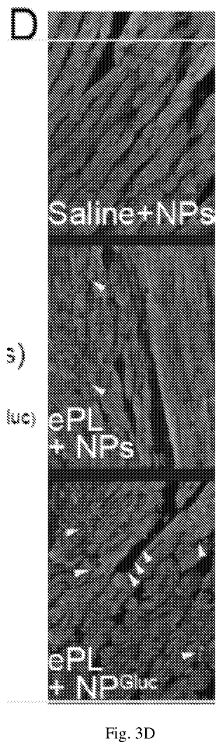

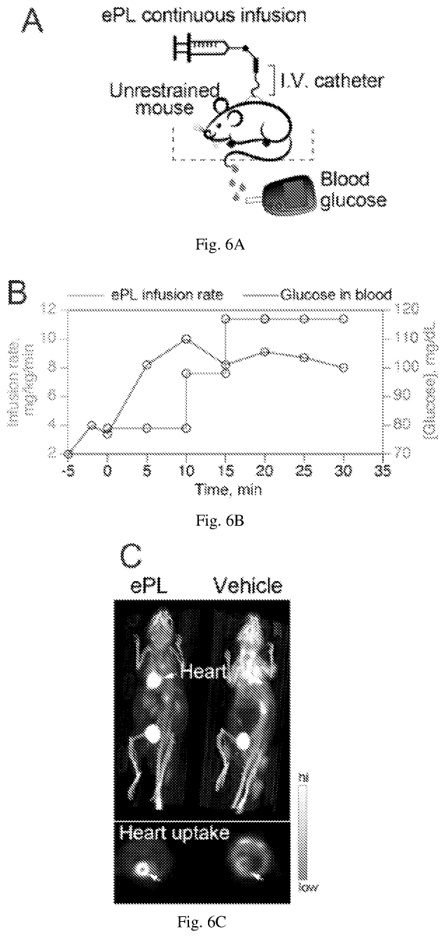

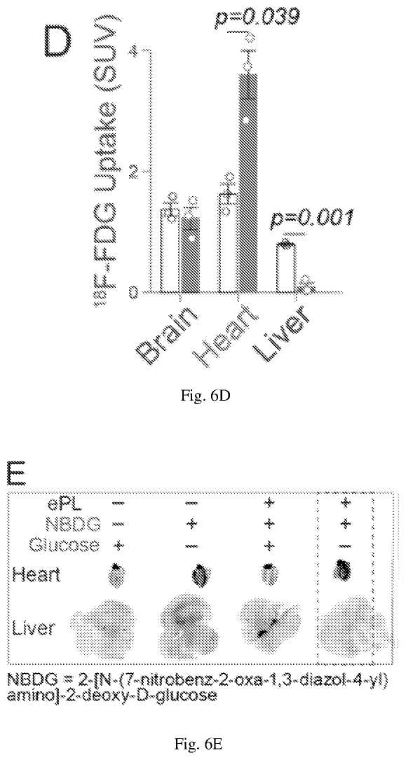

We demonstrate that ePL: a) increases specificity of delivery of various modalities to the heart and de-targets the liver (FIGS. 3-5); b) exhibits a new mechanism of action that is likely applicable to any delivery vehicle/entity, including nanoparticles, viruses, and cells (FIGS. 6-8).

### Biodistribution and Safety

ePL consists of a synthetic biodegradable polymer based on poly(L-lactic acid) and terminated by carboxyl groups. At molecular weight of about 120 kDa it forms mixed micelles in aqueous buffers ranging 100-400 nm in diameter. To understand ePL deposition in various organs after i.v. injection, we conjugated ePL to a Cy5 fluorescent dye and blended 5% of Cy5-labeled ePL with unlabeled ePL. After injection at 10 mg/kg in C57BL6 mice, perfusion, organ excision and imaging we found that 90% of ePL accumulated in the liver and 5% in the spleen. No ePL deposition in the heart, brain, or muscle was observed. The clearance of ePL from systemic circulation was very rapid (T_{1/2}=5.3±2.6 min), additionally suggesting ePL has high liver affinity. Because ePL is based on poly-lactide, which is highly biodegradable and considered safe, we did not expect significant toxicities associated with ePL. Examination of heart and liver sections from mice injected with 30 mg/kg ePL using histological staining revealed that these tissues appeared all normal following H&E staining, with no obvious sign of fibrosis or cell infiltrations. It is worth noting that a long-term, chronic in vivo use of ePL is a highly unlikely scenario for one-and-done therapies such as AAV gene delivery, as we will demonstrate below.

### Delivery of Model Nanoparticles, Fluorospheres

In one experiment performed in our laboratory aimed to investigate biodistribution of various nanoparticles and micelles administered at the same time, we observed an intriguing biodistribution pattern of model polystyrene latex fluorospheres, but only when simultaneously injected with ePL. We followed up on these initial findings using nanoparticle (NP) fluorospheres with a hydrodynamic size of 25 nm incorporating an exceptionally bright far red fluorophore. Optimization of the injection schedule (FIG. 3A) and surface coatings on fluorospheres revealed that administration of ePL 15 min before NPs significantly enhanced the NP deposition to the heart and de-targeted the liver (FIG. 3B). Other organs of the reticuloendothelial system (RES) were also de-targeted. The effect was particularly striking when glucose-bearing NPs were used (FIG. 3B, highlighted in dotted box). Glucose surface modification (glycosylation) was achieved through first covalent attachment of amino-PEG4-alkyne (CAS 1013921-36-2) to the surface of NPs using carbodiimide chemistry, followed by Cu-catalyzed click chemistry with 2-azido-2-deoxy-D-glucose (Az-DG, CAS 56883-39-7) resulting in glycosylated NPs (NP^{Gluc}). Control NPs were amino-PEG4-alkyne-functionalized, but quenched with NaN₃ instead of the reaction with Az-DG.

Notably, ePL+NP^{Gluc} demonstrated up to an order of magnitude preference to the heart vs. liver (FIG. 3C) and were confirmed to accumulate within cardiomyocytes using immunofluorescence in heart sections (FIG. 3D). In sum, ePL redirects NP entities to the heart while de-targeting the liver and RES. Such targeting appears to depend on glycosylation and is hypothesized to be applicable to other targeting modalities/vehicles.

Delivery of adeno-associated viruses (AAVs)

Encouraged by the preliminary results on ePL-enhanced NP delivery and safety, we set out to expand the portfolio of possible delivery vehicles. For our pilot studies, we selected serotype AAVrh74. because it has been clinically validated using intravenous administration and it is considered to have skeletal and cardiac muscle tropism. We wanted to test if ePL would further increase AAVrh.74 affinity to the heart.

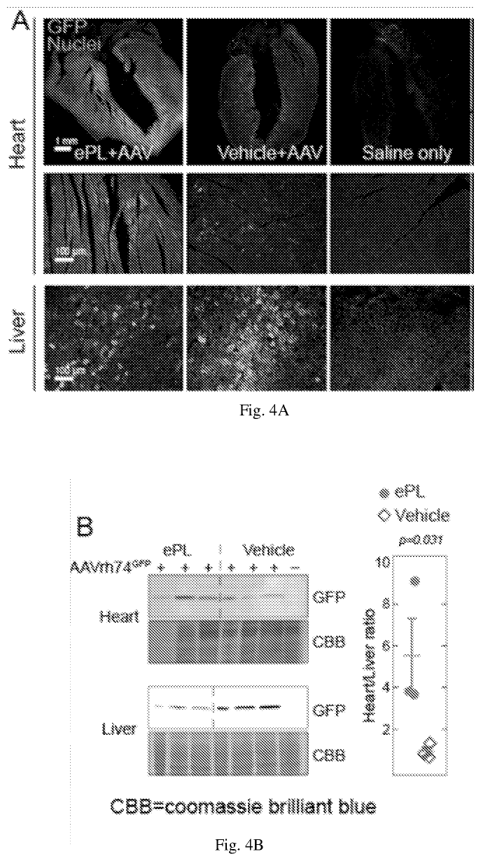

Moreover, most recombinantly-produced AA Vs possess intrinsic surface glycosylation,⁴ advantageous for targeting with ePL (FIG. 3). A single injection of 5e11 μg/kg single-stranded AAVrh74 carrying the eGFP transgene under the control of the CMV promoter (referred to as AAVrh.74 further in the text for simplicity) was performed following the injection schedule similar to that in FIG. 3A. Because the expression of the AAV-carrying transgene (eGFP in this case) becomes appreciable at four-to-six weeks following the delivery, the protein level of eGFP expression was examined in the heart and the liver five weeks after the ePL/AAV injection. Approximately a six-to-ten-fold increase in the eGFP expression in the heart was observed with ePL+AAV as compared to animals that received AAV alone (FIG. 4A, B). Biodistribution studies using qPCR in major organs confirmed heart targeting and liver de-targeting (FIG. 4C) and also demonstrated (although not statistically significant) brain and lung de-targeting. We estimated (based on the total DNA extracted and organ weight) that a total of 11% of injected AAV dose was delivered to the heart, which is in stark contrast to 0.2-2% delivery reported in the literature, even for serotypes with the highest heart affinity (e.g., AAV9). Strikingly, the AAV1 serotype, which is known for its poor heart tropism in i.v.-injected mice, demonstrated a significant 14.1-fold increase in heart delivery with ePL vs. AAV1-only control (FIG. 4D). In the AAV1-only group, 3 of 5 injected animals did not show any viral DNA in the heart (N.d.), whereas high liver accumulation was detected in the same animals. Interestingly, AAV1 was previously shown to have moderate heart tropism in rats injected intramyocardially. Collectively, this data suggests ePL-enhanced cardiac targeting of AAVs was not only due to liver and RES de-targeting, but also through a new heart-mediated mechanism. This is especially intriguing because ePL does not accumulate in the heart and is unlikely to interact with the viral particles directly considering that ePL persistence in the circulation (<<15 min) is far shorter than that of AAV serotypes tested in our studies.

### Mesenchymal Stem Cell (MSC) Delivery

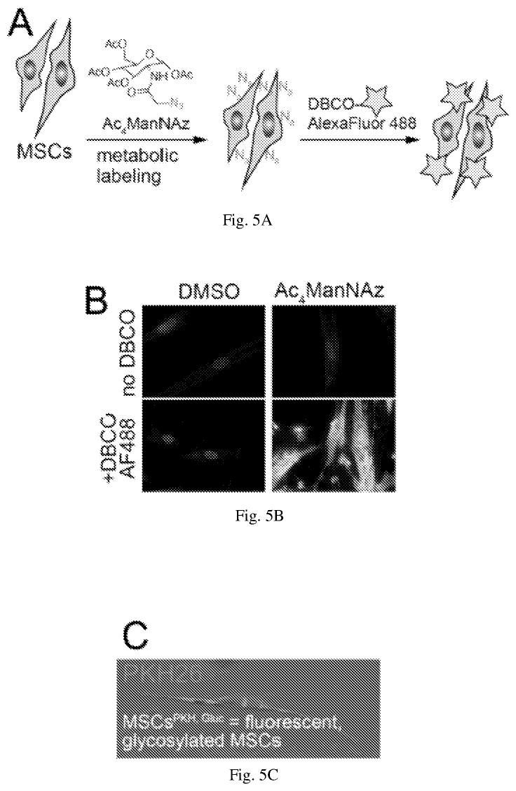

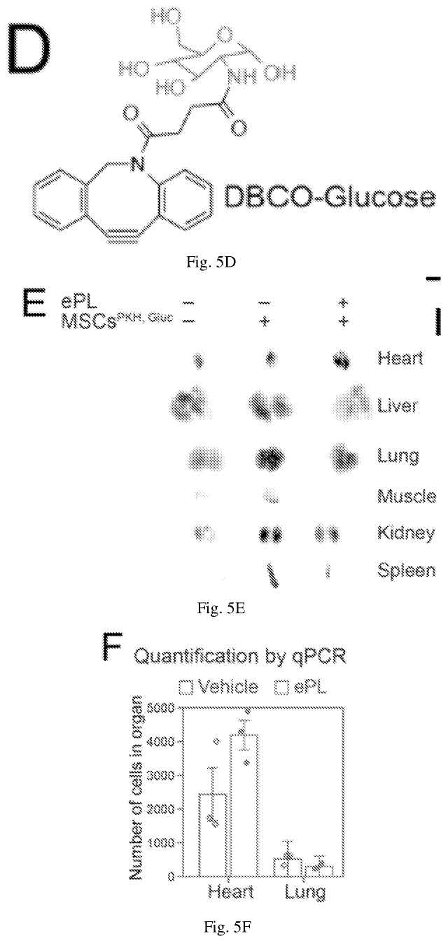

We were interested in testing a therapeutically-relevant entity that differs drastically from small-sized NPs and viruses (which are ˜20-25 nm in size). We selected MSCs because of their much larger size, extremely fast blood clearance (<5 min), and their accumulation preference for lung, rather than the liver. To enrich the cell surface with glycosylation, we employed a well-known metabolic labeling strategy. Metabolic labeling that allows cell surface modifications in live MSCs was accomplished by means of culturing MSCs in the presence of cell-permeable, intracellularly processed unnatural sugar N-α-azidoacetylmannosamine Ac₄ManNAz, that introduces azido groups on the cell surface (FIG. 5A,B). These azides can be further conjugated to a fluorophore (FIG. 5B) or glucose through a strain activated click-chemistry using dibenzocyclooctyne DBCO derivatives (FIG. 5C-D). For tracking, we labeled MSCs with PKH26 red fluorescent dye (FIG. 5C). Resulting MSC^{PKH, Gluc} were injected at 5×10⁵ cells/mouse, 15 min after ePL injection, similar to that in FIG. 5A. Two hours post injection (p.i.), excised organs were imaged or pulverized in liquid nitrogen for further analysis by qPCR. The data show heart targeting and liver and lung de-targeting (FIG. 5E) in animals pre-injected with ePL. A quantitative analysis of Alu sequences in the heart and lung using qPCR and a standard curve obtained from known MSC cell numbers demonstrated a trend in heart accumulation, though no statistical significance was achieved. The trend would likely become significant if a larger number of animals was used. Nevertheless, these results are encouraging and increase our confidence in the potential durability of the delivery effect, regardless of the nature of the delivery vehicle.

### ePL Decreases Glucose Uptake in the Liver but Increases in the Heart

According to the pilot data on the delivery of NPs, glycosylation of the vehicle enhances heart targeting. This opens a possibility of further improvement of the ePL system through tuning of the level of vehicle glycosylation. Given these considerations, we studied whether ePL has an effect on systemic blood glucose levels. In vivo glucose utilization studies were performed using an in-house developed protocol based on the modified workflow from hyperinsulinemic-euglycemic clamps. C57BL6 mice were surgically implanted with an i.v. catheter through which ePL was infused during the experiment (FIG. 6A). The experiment was conducted in conscious, unrestrained and minimally-stressed animals. The mice were able to freely move because the catheter was connected through a swivel. The blood samples were obtained from the tail snips at 5 min intervals prior to ePL infusion and immediately after the start of ePL infusion at variable rates using a syringe pump. Blood glucose was measured using a hand-held glucometer. In these experiments we observed a steady, yet saturable increase in the glucose levels upon ramping up the ePL infusion rates (FIG. 6B). In another experiment, the whole body glucose uptake was assessed using fluorodeoxyglucose (FDG)-positron emission tomography (PET). A single dose of ePL significantly increased 18F-FDG tracer uptake in the heart just 1 h after ePL administration, as compared to vehicle-injected animals (FIG. 6C, D). This occurred concomitantly with no changes in the FDG uptake in the brain, however, the uptake in other major glucose-consuming organs including the liver was significantly reduced (FIG. 6D). Finally, the uptake of fluorescent analogue of glucose, NBDG (1 mg/kg), was studied with and without ePL and in the presence of the large excess of unlabeled glucose (500 mg/kg). The data showed that even after extensive organ perfusion, NBDG fluorescence was highly localized in the heart of animals pre-injected with ePL (FIG. 6E), however was diminished in competition experiments with unlabeled glucose. The data above suggest an opposing action of ePL on glucose uptake in the heart and liver. We additionally confirmed attenuated hepatic glucose uptake in mice intraperitoneally injected with ePL using traditional hyperinsulinemic-euglycemic clamps (not shown). These data are supportive of the preferential heart delivery of vehicles/entities that are glycosylated. Moreover, the enhanced uptake of NBDG with ePL is yet another example of an entity that can be delivered with high selectivity to the heart, i.e., a small molecule. A fluorophore in this case, but potentially, any pharmacologic agent that can be glucose-conjugated without the loss of therapeutic activity, is likely to improve heart targeting with ePL.

### ePL Increases Circulation Half-Life of AAV9

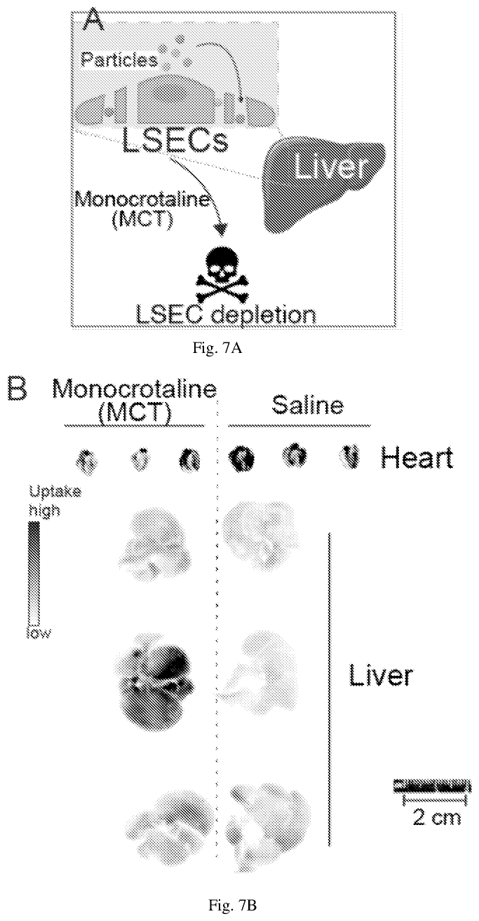

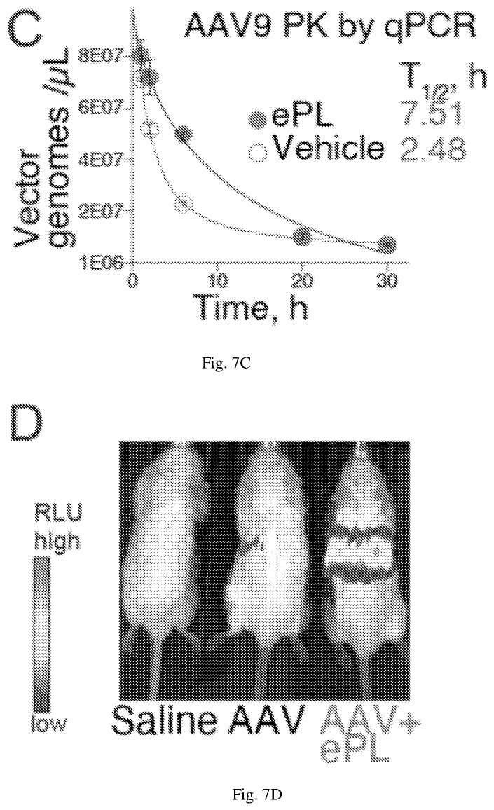

The findings shown above only partially explain the enhanced NPs/AAV heart uptake and liver de-targeting when ePL is used with NPs/AAVs. It is well known that liver sinusoidal endothelial cells (LSECs) are endocytic and very efficient at clearing circulating particles and viruses through a variety of surface-expressed receptors. We depleted LSECs in C57BL6 mice (FIG. 7A) using two i.p. injections of monocrotaline (MCT) over 48 h as previously described. Next, ePL and NP^{Gluc} fluorospheres were injected as described in Delivery data. Strikingly, the animals pre-injected with MCT (LSECs depleted) demonstrated diminished heart NP^{Gluc} uptake and high liver accumulation (FIG. 7B). This data suggests that LSECs are important for ePL uptake. It is possible that ePL “overwhelms” functional LSECs that would be normally responsible for clearance of NPs and AAVs, thus prolonging their persistence in circulation, and allowing more time to target the heart.

When LSECs are depleted, ePL and NPs/AAVs are likely cleared by liver Kupffer cells, which may have a different mechanism and kinetics of uptake. Finally, it has been proposed that intrinsically high circulation half-life of AAV9, a serotype with the best-in-class heart transduction efficacy, may be a factor that allows to overcome slow transvascular AAV9 transport through the tightly sealed capillary endothelium in the heart, thus efficiently transducing cardiomyocytes by virtue of a substantially longer circulation time. PK studies using ePL co-administered with 1e10 vg/kg AAV9 demonstrated a three-fold increase in circulation half-life with ePL as compared to vehicle-injected mice (FIG. 7C). Further supporting our hypothesis above, FVB/N mice injected with AAV9 carrying luciferase transgene demonstrated much higher (16.7 fold) overall levels of luciferase expression when pre-injected with ePL (FIG. 7D). We posit that any AAV serotype can be enhanced through prolonging its half-life with ePL, even without serotype engineering.

ePL Increases the Expression of Glycosylated Residues, which Predict AAV Targeting Tropism

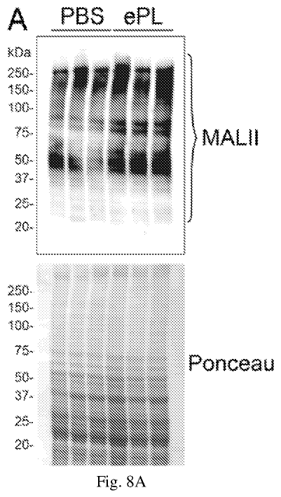

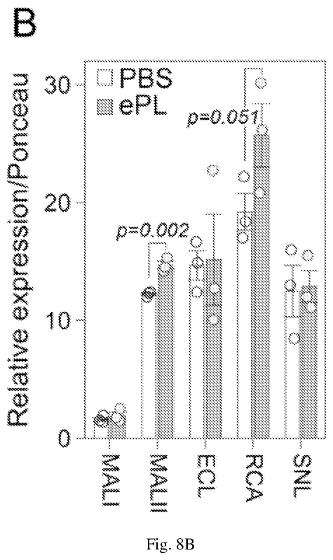

One of the major targeting mechanisms by which AAV particles target the tissue is through the interaction with various carbohydrates on the cell surface. To understand whether ePL is able to change carbohydrate landscape in the heart and thus predict the tropism of certain AAV serotypes, we performed a series of experiments aimed to show the expression of glycans and sialic acid residues in the heart. WT mice were injected with a bolus ePL (30 mg/kg) or PBS vehicle control and 1 h later the mice were euthanized and the hearts were excised. Heart homogenates were analyzed using Western blotting using various lectins that have high affinity for different carbohydrate residues (FIG. 8). Thus, the hearts from ePL-injected animals demonstrated statistically significant increase in binding of MALII lectin (Maackia Amurensis) that binds to sialic acid in an (α-2,3) linkage. Similarly, RCA lectin (Ricinus Communis Agglutinin), which binds to galactose or N-acetylgalactosamine also demonstrated enhanced affinity to ePL heart samples. According to the literature, N-linked α-2,3 sialic acid predicts high tropism for AAV1 and AAV5, whereas galactose expression predicts tropism for AAV9. The heighted expression of these glycosylated residues is likely due to enhanced glucose uptake in the heart; where this glucose serves as a precursor in enhanced synthesis of glycosylated proteins.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-63. (canceled)

64: A method of promoting accumulation and/or uptake of a therapeutic agent and/or diagnostic agent in a heart of a subject in need thereof, the method comprising:
systemically administering to the subject a therapeutic agent and/or diagnostic agent and an amount of a composition including a plurality of poly(L-lactic co-glycolic acid) (PLLGA) nanoparticles effective to promote accumulation and/or uptake of the therapeutic agent and/or diagnostic agent in the heart, wherein the therapeutic agent and/or diagnostic agent is not encapsulated by or conjugated to the PLLGA nanoparticles.

65: The method of claim 64, wherein the PLLGA nanoparticles have an average diameter of about 50 nm to about 600 nm.

66: The method of claim 64, wherein the PLLGA of the PLLGA nanoparticles has a molecular weight of about 15 kDa to about 200 kDa.

67: The method of claim 64, wherein the PLLGA comprises about 60% L-lactic acid to about 85% L-lactic acid.

68: The method of claim 67, wherein the PLLGA comprises about 40% glycolic acid to about 15% glycolic acid.

69: The method of claim 64, wherein the PLLGA nanoparticles comprise PLLGA micelles.

70: The method of claim 64, wherein the PLLGA nanoparticles are formulated from PLLGA, a solvent or liquid carrier, and a surfactant.

71: The method of claim 64, wherein the composition is administered to the subject at an amount effective to inhibit systemic uptake of the therapeutic and/or diagnostic agent in organs other than the heart.

72: The method of claim 71, wherein the organs other than the heart include at least one of the brain, non-cardiac muscle, liver, lung, and adipose tissue.

73: The method of claim 64, wherein the therapeutic agent comprises at least one of a small molecule, nanoparticle, virus, nucleotide, peptide, protein, antibody or antigen binding fragment thereof, and/or cell.

74: The method of claim 64, wherein the therapeutic agent and/or diagnostic agent is naturally or synthetically glycosylated and/or includes, is complexed with, and/or conjugated to glucose or a glucose analog.

75: The method of claim 64, wherein the therapeutic agent comprises a viral vector.

76: The method of claim 64, wherein the therapeutic agent comprises an adeno-associated viral vector.

77: The method of claim 64, wherein the adeno-associated viral vector comprises at least one of AAV1, AAV2, AAV6, AAV8, AAV9, AAVrh74, AAVrh10, AAV5, AAV7, AAVS3, AAVHSC, AAV2.7m8, AAV-LK03, AAV8/Olig001, AAV2i8, AAVhu37, AAV2tYF, AAVh1, AAVhu68, AAVrh.8, AAVrh9, AAV.PHP.B, AAV.PHP.eB, AAV.PHP.S, AAV/BBB, AAV-DJ, AAVr3.45, AAV-sh10, AAV2(Y444F), AAV4, AAV-RPF2, or AAV3b.

78: The method of claim 75, wherein the viral vector includes cDNA that encodes at least one of Titin, Lamin-A/C, Myosin 7 Heavy Chain, Myosin 6 Heavy Chain, Sodium Channel Protein Type 5 alpha subunit, Cardiac-Type Myosin Binding Protein C, Cardiac Muscle Troponin T, RNA-Binding Protein 20, Cardiac Troponin I, Regulatory Light Chain of Cardiac Myosin beta, Myosin Light Chain Ventricular Isoform, Plakophilin 2, Desmoplakin, Desmoglein, LAMP2B, Desmocolin 2, Junction Placoglobin, adenylyl cyclase (AC) 6 (AC6), sarco/endoplasmic reticulum (SR) Ca²⁺-ATPase (SERCA2a), SUMO1, S100A1, I1c, VEGF-A, VEGF-B, β-adrenergic receptor kinase-ct, urocortins, B-cell lymphoma 2 (Bcl2)-associated anthanogene-3 (BAG3), Heme Oxygenase-1, anti-fribrotic agents, anti-inflammatory agents, or anti-hypertrophic agents.

79: The method of claim 64, wherein the therapeutic agent comprises a plurality of cells, such as mesenchymal stem cells.

80: The method of claim 64, wherein the diagnostic agent includes at least one of an inorganic tracer, radio metal ions, small organic tracers, or radiometal complex tracers and wherein the diagnostic agent is naturally or synthetically glycosylated and/or includes, is complexed with, and/or conjugated to glucose or glucose analog.

81: The method of claim 64, wherein the subject has or is at risk of a cardiac disease or disorder.

82: The method of claim 81, wherein the cardiac disease or disorder comprises at least one of Heart failure with reduced ejection fraction (HFrEF), Heart failure with preserved ejection fraction (HFpEF), Heart failure with mid-range ejection fraction (HFmrEF), hypertrophic cardiomyopathy (HCM), arrhythmogenic right ventricular cardiomyopathy (ARVC), dilated cardiomyopathy (DCM), diabetic cardiomyopathy (DbCM), Brugada Syndrome, Barth syndrome, arrhythmogenic left ventricular cardiomyopathy (LDAC or ALVC), Ventricular fibrillation, Ventricular tachycardia, Left Ventricular Non Compaction (LVNC), catecholaminergic polymorphic ventricular tachycardia (CPVC), Paroxysmal familial ventricular fibrillation (PFVF), Naxos disease, Carvajal syndrome, Wolff-Parkinson-White syndrome, Fabry disease, LEOPARD syndrome, Noonan syndrome, Anderson-Fabry disease, Familial amyloidosis, Kearns Sayre syndrome, MELAS syndrome, Becker MD, Duchenne MD, Emery-Dreifuss/Limb-Girdle MD, Friedreich's ataxia, Myotonic dystrophy, Down syndrome, AMPK mediated glycogenic storage, Pompe disease, Danon disease, Niemann-Pick, Refsum disease, or Chagas disease.

83: The method of claim 64, wherein the therapeutic agent and/or diagnostic agent is administered to the subject within 6 hours, within 5 hours, within 4 hours, within 3 hours, within 2 hours, or simultaneously with administration of the composition.
